(19) United States
(12) Patent Application Publication
(10) Pub. No.: US 2022/0415487 A1
(43) Pub. Date: Dec. 29, 2022

(54) EMERGENCY ROOM KIOSK FOR FINANCIAL ASSISTANCE AND INFORMATION

(71) Applicant: Hector Solis, Harlingen, TX (US)

(72) Inventor: Hector Solis, Harlingen, TX (US)

(21) Appl. No.: 17/362,898

(22) Filed: Jun. 29, 2021

(51) Int. Cl. G16H 40/20; G06Q 50/22; A61L 2/24; A61L 2/00

(52) U.S. Cl. CPC G16H 40/20; G06Q 50/22; A61L 2/24; A61L 2/0088; A61L 2202/14

(57) ABSTRACT

A network-connected information and registration kiosk for use in waiting rooms of medical facilities such as Emergency Rooms configured to facilitate and expedite the check-in procedure, as well as the assist in a user's search for, and registration to, financial assistance programs. The kiosk presents an easy-to-use user interface which enables the user to locate and apply to assorted programs designed to assist in the payment for medical services, treatment, and procedures in accordance with his or her visit to the medical facility. The kiosk enables the user to get a jump-start on qualifying for such financial assistance programs immediately after the check-in process at the medical facility.

### FIELD OF THE PRESENT INVENTION

The present invention relates to the field of network-connected support devices, and more specifically relates to a computer-based platform, embodied in a kiosk, configured to facilitate and expedite the eligibility determination and application process at an Emergency Room at a hospital or similar medical facility while allowing users to search and attempt to qualify for financial assistance for sought medical procedures, visits, and treatments.

### BACKGROUND OF THE PRESENT INVENTION

Upon experiencing a medical emergency, most people are required to visit the Emergency Room (ER) of their local hospital. The ER provides a convenient location for individuals to have their medical emergency addressed, as adequate medical personnel are generally present at all times to facilitate the treatment of a wide variety of medical emergencies. However, the process is not always timely. The check-in process frequently requires people to wait just to enter the system so that they may then wait in the waiting room to be seen by medical professionals. Sometimes, this wait can take hours, especially in large metropolitan areas or at peak times.

Often, the ER is used by individuals who do not necessarily have a dire medical emergency, not because they do not wish to be seen, but because the often have no other recourse due to a lack of available insurance or funding to be seen by a specified provider outside of the ER. As a result, patients of the ER often have a good bit of time on their hands, otherwise left idle to read, listen to music, or other similar activities. However, this time could be better spent attempting to qualify for a variety of financial assistance programs which are available.

Unfortunately, most individuals are not aware that these programs exist, let alone whether or not they qualify. They also generally do not know how to apply for these programs. Additionally, there are presently no easy means by which users may apply or determine their qualification status for such programs from within the hospital itself. Many patients end up trying to apply for coverage many days or months later, after their ER visit is long over, ultimately delaying the application process. Additionally, these patients are then sent a bill which can be shocking without knowing their options. If there were an expedient method and system by which patients could be checked-in and could then immediately begin the application and qualification process for financial assistance for their medical expenses, more patients would be capable of covering their incurred expenses, and time would be better managed upon arrival at the ER.

Thus, there is a need for a new system and platform on which users may determine their eligibility and attempt to qualify for financial assistance programs within the waiting room of the ER. Such a system preferably employs a medical kiosk configured to enable the user to safely inquire about aid programs, and determine if he or she may qualify. Additionally, such a system preferably enables the patients to apply to pertinent programs while they wait to be seen.

### SUMMARY OF THE PRESENT INVENTION

The present invention is a kiosk platform and system for use in an Emergency Room waiting area setting or similar location of a medical facility. The platform is configured to enable patients to check-in to the kiosk, receive pertinent registration credentials, view relevant information relating to their visit, and to apply to programs designed to provide financial assistance for medical expenses. The platform provides easy-to-follow prompts that guide the patient through the check-in process, and then asks the patient a series of questions which will help to determine whether or not he or she should pursue any or all potential programs for financial assistance for his/her medical needs. The kiosk is configured to self-sanitize between patients, and additionally dispenses sanitizer to the patient automatically upon approach of the kiosk. Additionally, the kiosk is able to print a unique identifier for the patient's use while in the hospital.

The following brief and detailed descriptions of the drawings are provided to explain possible embodiments of the present invention but are not provided to limit the scope of the present invention as expressed herein this summary section.

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

The present invention will be better understood with reference to the appended drawing sheets, wherein:

FIG. 1 depicts a view of the emergency room kiosk of the present invention as seen from the front.

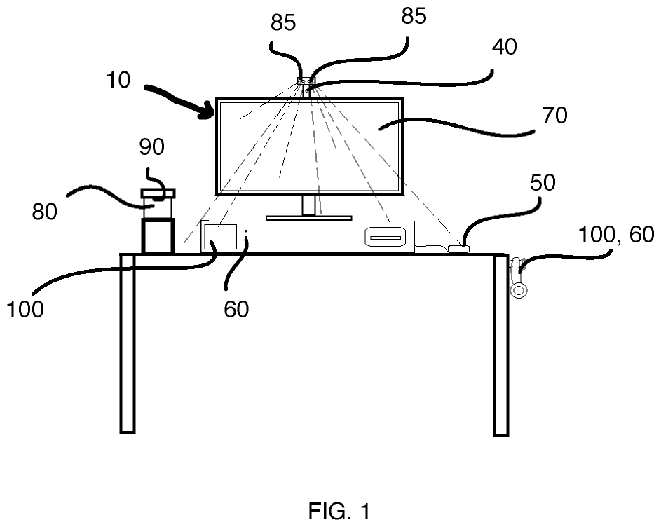

FIG. 2 exhibits an example of the room layout employed for hosting the kiosk system of the present invention as seen from above.

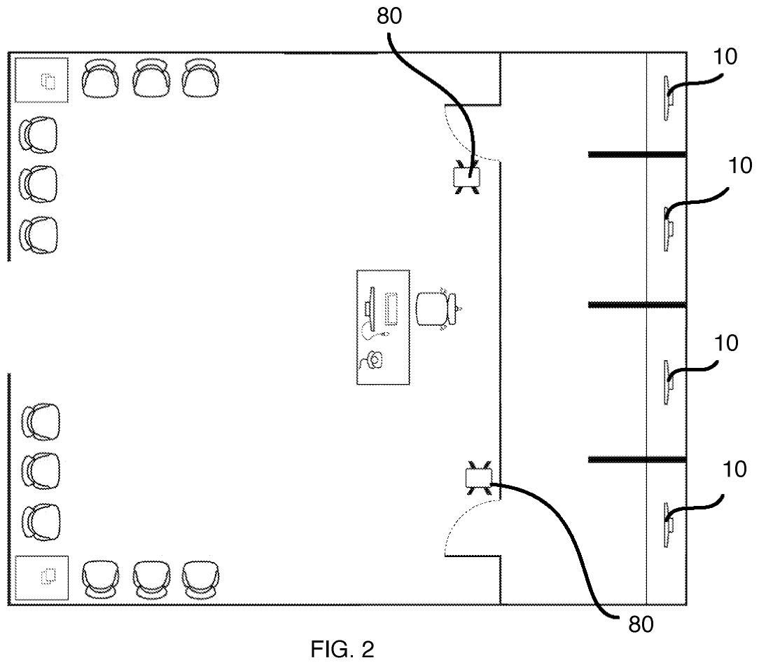

FIG. 3 is a flow chart detailing the process of use of the system of the present invention by a user.

FIG. 4 depicts a flow chart detailing the patient's use of the kiosk of the present invention in a basic form, including creating an application for aid.

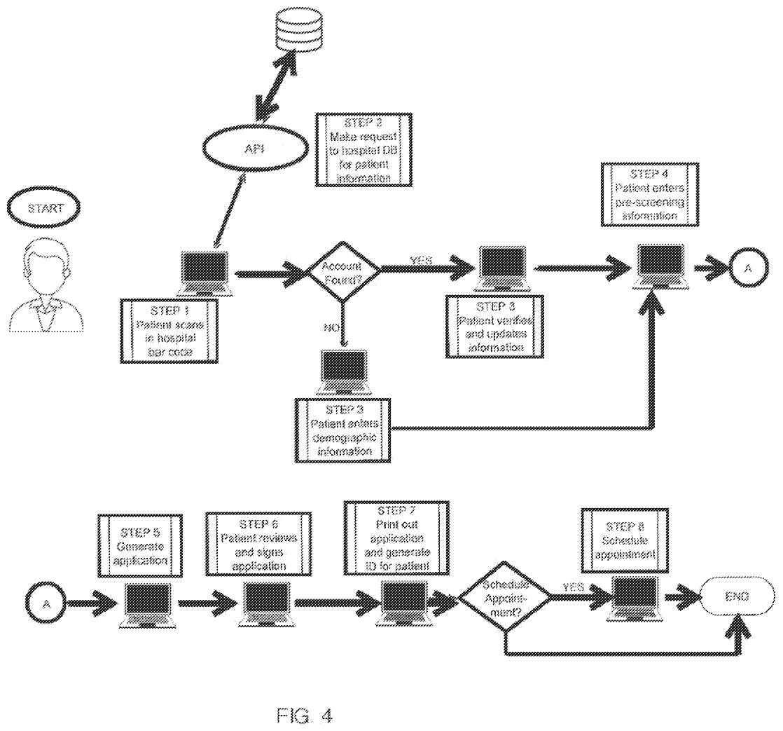

### DETAILED DESCRIPTION OF THE PREFERRED EMBODIMENT

The present specification discloses one or more embodiments that incorporate the features of the invention. The disclosed embodiment(s) merely exemplify the invention. The scope of the invention is not limited to the disclosed embodiment(s).

References in the specification to “one embodiment,” “an embodiment,” “an example embodiment,” etc., indicate that the embodiment described may include a particular feature, structure, or characteristic, but every embodiment may not necessarily include the particular feature, structure or characteristic. Moreover, such phrases are not necessarily referring to the same embodiment, Further, when a particular feature, structure, or characteristic is described in connection with an embodiment, it is submitted that it is within the knowledge of one skilled in the art to effect such feature, structure, or characteristic in connection with other embodiments whether or not explicitly described.

The present invention is a platform and method of use thereof for the registration and/or checking-in of patients of an Emergency Room of a hospital or similar medical facility. The platform is present on an easy-to-use kiosk (10). The kiosk (10) of the present invention is equipped with a computer (20), printer (30), camera (40), input mechanism (50), microphone (60), speaker (100), and a screen (70). Additionally, the kiosk (10) is preferably equipped with a sanitizer mist dispenser (80) which is configured to automatically spray the surface of the input mechanism (50) and/or screen (70) of the kiosk (10) upon the approach of a patient to the kiosk (10), sanitizing the system for use between patients. Similarly, a hand sanitizer dispenser (80), equipped with a motion detector (90) is configured to dispense sanitizer onto the hand(s) of the patient when he or she places a hand in range of the motion detector (90). A sanitizer mist sprayer (85) is also configured to enable the kiosk to self-sanitize between uses.

The screen (70) of the present invention may be a conventional capacitive touch screen in order to function as the input mechanism (50), however a keyboard may also be present as an input mechanism (50). The screen (70) is configured to display text in multiple languages in accordance with the language preference of the patient. The screen (70) guides the patient through the check-in process (for checking into the kiosk itself) and prompts the patient to input his or her personal information. Personal information preferably includes the patient's name, address, date of birth, reason for visit, and known medical history. In the event that the patient wants help filling out the information, a video call may be instantly made to an onsite or offsite representative to assist the patient with use of the kiosk (10). The video call is facilitated by the camera (40), speaker (100), and microphone (60) of the present invention. Some embodiments of the present invention may be equipped with a headset to ensure the privacy of the patient during the video call.

After checking in to the ER via the kiosk (10), the computer (20) instructs the printer (30) to print a unique identifier for the patient which may be used by medical personnel, as well as by the kiosk (10) for later use via the camera (40), which is preferably configured to scan and recognize the unique identifiers to enable the user to rapidly access the features of the kiosk upon his or her return.

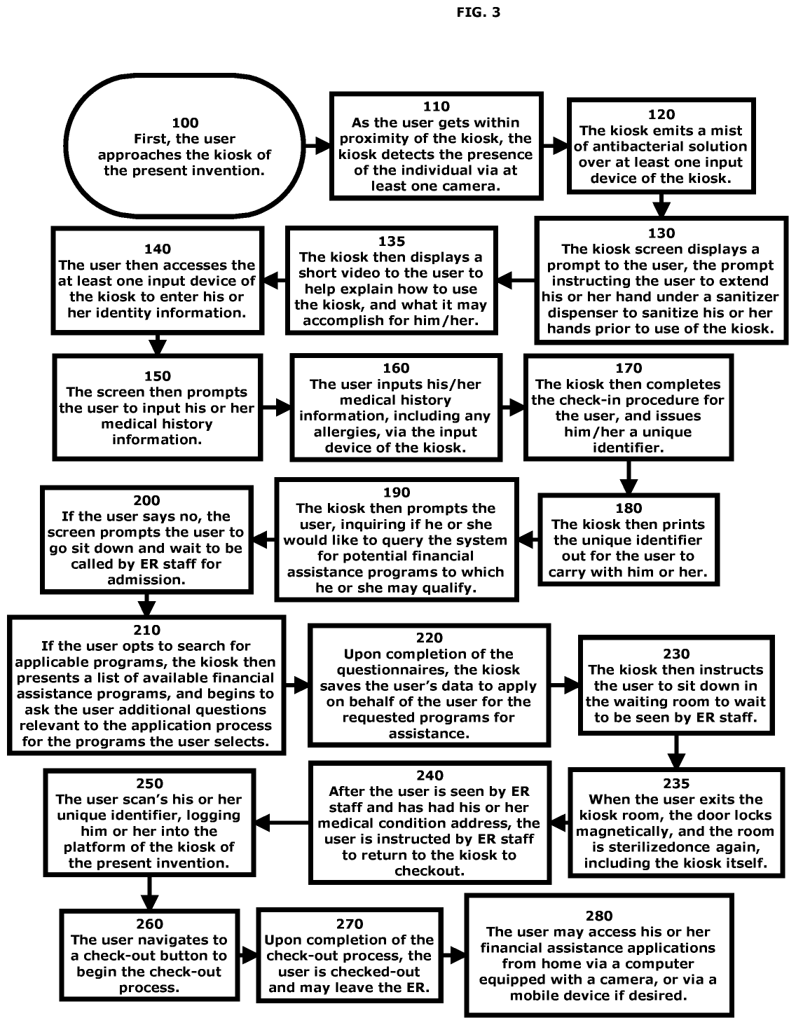

The process of use of the system and platform of the present invention, as detailed in FIG. 3, is preferably as follows:
1. First, the user approaches the kiosk of the present invention. (100). The kiosk is preferably contained in a small room equipped with a door. The door is magnetically locked, and is configured to open only for the user once admitted to the room for access to the kiosk.
2. As the user gets within proximity of the kiosk, the kiosk detects the presence of the individual via at least one camera. (110)
3. The kiosk emits a mist of antibacterial solution over at least one input device of the kiosk. (120) This may occur prior to the individual entering the room, such as immediately after the previous user exits the room. During the sterilization misting process, the room is preferably locked.
4. The kiosk screen displays a prompt to the user, the prompt instructing the user to extend his or her hand under a sanitizer dispenser to sanitize his or her hands prior to use of the kiosk. (130)
5. The kiosk then displays a short video to the user to help explain how to use the kiosk, and what it may accomplish for him/her. (135)
6. The user then accesses the at least one input device of the kiosk to enter his or her identity information. (140). The information preferably includes the following: a birth date of the user, a first name of the user, a last name of the user, race of the user, and nationality of the user.
7. The screen then prompts the user to input his or her medical history information. (150)
8. The user inputs his/her medical history information, including any allergies, via the input device of the kiosk. (160)
9. The kiosk then completes the check-in procedure for the user, and issues him/her a unique identifier. (170)
10. The kiosk then prints the unique identifier out for the user to carry with him or her. (180). The unique identifier may be a barcode, QR code, or similar identifier, and is preferably used when the user wishes to revisit the kiosk to check-out, to continue a financial assistance application, or when he or she is eventually seen by hospital staff. It should be noted that the unique identifier does not necessarily replace the need for a hospital bracelet once the user is admitted.
11. The kiosk then prompts the user, inquiring if he or she would like to query the system for potential financial assistance programs to which he or she may qualify. (190)
12. If the user says no, the screen prompts the user to go sit down and wait to be called by ER staff for admission. (200)
13. If the user opts to search for applicable programs, the kiosk then presents a list of available financial assistance programs, and begins to ask the user additional questions relevant to the application process for the programs the user selects. (210)
14. Upon completion of the questionnaires, the kiosk saves the user's data to apply on behalf of the user for the requested programs for assistance. (220)
15. The kiosk then instructs the user to sit down in the waiting room to wait to be seen by ER staff. (230)
16. When the user exits the kiosk room, the door locks magnetically, and the room is sterilized once again, including the kiosk itself. (235)
17. After the user is seen by ER staff and has had his or her medical condition address, the user is instructed by ER staff to return to the kiosk to checkout. (240)
18. The user scan's his or her unique identifier, logging him or her into the platform of the kiosk of the present invention. (250)
19. The user navigates to a check-out button to begin the check-out process. (260)
20. Upon completion of the check-out process, the user is checked-out and may leave the ER. (270)
21. The user may access his or her financial assistance applications from home via a computer equipped with a camera, or via a mobile device if desired. (280). This is helpful in the event that the user did not have enough time to apply for all the programs for which he or she may qualify, or if he or she wishes to amend any information input during the check-in process.

It should be noted that in the preferred embodiment of the present invention, the user may opt to initiate a video call with a representative from the kiosk via the video camera. The representative may assist the user in navigating the kiosk, or guide the user through the entire process of use of the present invention. Similarly, the representative may interview the user through the video call and enter his/her input manually to the kiosk remotely if necessary.

In some embodiments of the present invention, the kiosk is preferably directly connected to the internet. However, in the preferred embodiment of the present invention, the kiosk is solely connected to an internal network of the medical facility, and only has indirect access to online links. The accessible links are preferably solely configured to facilitate the application of the patient to the financial assistance programs, and are preferably displayed on the screen (70) within a locked browser, preventing the patient from visiting external sites during use of the kiosk (10).

Further, it should be noted that the system of the present invention may incorporate a mobile device application, available on major application distribution channels such as the Apple™ AppStore™, and Google™ PlayStore™, and other similar application stores. The mobile device application is preferably capable of providing the user of the kiosk of the present invention a portal to access his or her information and financial assistance application forms from his or her mobile device after the ER visit. Coding of both the mobile device application and the software of the kiosk (10) of the present invention is preferably developed with the patient in mind such that the patient is adequately assisted, and such that the official document required for processing the financial assistance claims are securely stored and accessible to the end user as applicable. It should be understood that the software of the present invention is equipped with advanced encryption configured to ensure the privacy of patients. A username and password may be employed across both the kiosk (10) and the mobile device application.

Additionally, it should be noted that the kiosk (10) of the present invention is preferably configured to employ remote storage to ensure the security of client/patients, including their personal and private data. Tamper-evident equipment is preferably present on the kiosk (10) to easily and readily indicate if the kiosk (10) has been opened and/or tampered with. Some embodiments of the kiosk (10) of the present invention may be equipped with an internal paper shredder configured to process the destruction of unwanted client documents.

Having illustrated the present invention, it should be understood that various adjustments and versions might be implemented without venturing away from the essence of the present invention. Further, it should be understood that the present invention is not solely limited to the invention as described in the embodiments above, but further comprises any and all embodiments within the scope of this application.

The foregoing descriptions of specific embodiments of the present invention have been presented for purposes of illustration and description. They are not intended to be exhaustive or to limit the present invention to the precise forms disclosed, and obviously many modifications and variations are possible in light of the above teaching. The exemplary embodiment was chosen and described in order to best explain the principles of the present invention and its practical application, to thereby enable others skilled in the art to best utilize the present invention and various embodiments with various modifications as are suited to the particular use contemplated.

I claim:

1. A registration, information, and financial aid assistance system for use by a patient in a medical facility comprising:
a kiosk, said kiosk disposed within a medical facility;
a computer, said computer housed within said kiosk;
a screen, said screen disposed in communication with said computer;
at least one input mechanism, said at least one input mechanism disposed in communication with said computer;
at least one sanitizer dispenser, said at least one sanitizer dispenser disposed in communication with said kiosk;
a printer, said printer disposed in communication with said kiosk;
a camera, said camera disposed in communication with said computer;
a microphone, said microphone disposed in communication with said computer;
wherein said computer is connected to the internet;
wherein said computer is connected to a local network of the medical facility;
at least one motion sensor, said at least one motion sensor disposed in communication with said computer and said at least one sanitizer dispenser;
wherein said at least one sanitizer dispenser is configured to dispense sanitizer when triggered by movement detected by said at least one motion sensor;
wherein said computer is configured to connect the patient to staff for assistance via a video call enabled via said camera and said microphone; and
wherein said computer is configured to enable the patient to apply for financial assistance for medical services while in the medical facility.

2. The apparatus of claim 1, wherein said sanitizer dispensed by said at least one sanitizer dispenser is in the form of mist.

3. The apparatus of claim 1, wherein said screen is a touch screen.

4. The apparatus of claim 1, wherein said at least one input device is at least one of the following: a mouse, a keyboard, and a touch screen.

5. The apparatus of claim 1, wherein said printer is configured to print a unique identifier pertaining to the patient for identification purposes.

6. The apparatus of claim 5, wherein said unique identifier is at least one of the following: a unique barcode, a unique QR code, and a unique numerical key ID.

7. The apparatus of claim 6, wherein said unique identifier may be read by said camera to facilitate reuse of said computer by the patient.

8. The apparatus of claim 1, wherein said screen is configured to display text generated by said computer in multiple languages in accordance with language preferences of the patient.

9. A method of checking into a medical facility and applying for financial aid for a patient via a kiosk comprising:
the patient approaching a self-service kiosk in the medical facility;
wherein the kiosk is a computer equipped with a screen, at least one input mechanism, at least one camera, at least one microphone, and at least one speaker;
a motion sensor detecting the motion of the patient as the patient nears the kiosk;
a sanitizer dispenser automatically dispensing hand sanitizer to the patient;
a screen of the kiosk illuminating and displaying a welcome video to the patient;
wherein the welcome video teaches the patient how to use the kiosk;
the screen of the kiosk prompting the patient to access at least one input device of the kiosk to input his or her identity information;
the kiosk saving the identity information to a database disposed in communication with the kiosk;
the kiosk prompting the patient to initiate a video call for assistance;
the kiosk prompting the user to input medical history information;
the kiosk saving the medical history information to the database;
the kiosk printing a unique identifier via a printer for the user;
wherein the unique identifier can be used to quickly log-in to the kiosk later;
the kiosk providing a list of potential financial assistance programs to which the patient is qualified to apply;
the kiosk walking the patient through the steps required to apply for financial assistance programs associated with medical care of the patient;
the patient applying for one or more programs;
the patient logging out of the kiosk; and
the patient waiting to be called for his/her medical examination or treatment.
